(19) United States
(12) Patent Application Publication
(10) Pub. No.: US 2025/0295131 A1
(43) Pub. Date: Sep. 25, 2025

(54) METHOD FOR PRODUCING FOOD PRODUCTS COMPRISING SULFUR-COMPRISING PROTEIN

(71) Applicant: PERFECT DAY, INC., Berkeley, CA (US)

(72) Inventors: Ty Wagoner, Oakland, CA (US); Suvash Kafley, Colleyville, TX (US); Poonam Bajaj, Castro Valley, CA (US); Ravirajsinh Jhala, Emeryville, CA (US); Sarah Leslie, San Pablo, CA (US)

(21) Appl. No.: 18/863,607

(22) PCT Filed: May 10, 2023
(86) PCT No.: PCT/US2023/021753

§ 371 Date: Nov. 6, 2024

Related U.S. Application Data
(60) Provisional application No. 63/340,347, filed on May 10, 2022.

(51) Int. Cl. A23C 9/152; A23G 9/40; A23J 3/08; A23L 2/66

(52) U.S. Cl. CPC A23C 9/1526; A23G 9/40; A23J 3/08; A23L 2/66

(57) ABSTRACT

The present invention relates generally to food products comprising proteins comprising sulfur-comprising amino acids, and to methods for producing such food products.

### RELATED APPLICATIONS

This application claims priority to U.S. Provisional Patent Application Ser. No. 63/340,347, filed on May 10, 2022, which is incorporated herein by reference, in its entirety.

### FIELD OF THE INVENTION

The present invention relates generally to food products comprising proteins comprising sulfur-comprising amino acids, and to methods for producing such food products.

### BACKGROUND OF THE INVENTION

Animal-derived food products (e.g., meat, milk, egg) are popular sources of nutrition. They may comprise high-quality protein, essential minerals (e.g., calcium, phosphorus, zinc, magnesium), and/or vitamins (e.g., riboflavin, vitamin A, vitamin B12). In addition, many such food products possess advantageous functional characteristics that permit production of a wide variety of derivative food products (e.g., yogurt, cheese, cream, ice cream, butter), and that are useful in industrial applications (e.g., production of polymers, therapeutics, household products).

However, animal-derived food products comprise components (e.g., lactose, allergens, saturated fats, cholesterol) that can cause unhealthy reactions in humans, and that are not easily avoided in the course of conventional, animal-based production processes. Moreover, production of these food products involves animal husbandry, which has a significant impact on animal welfare and the environment, and which bears the potential for contamination with pesticide residues, heavy metals, aflatoxin M1, and pathogens.

These concerns have fueled development of alternatives to animal-derived food and other products (e.g., cosmetics, personal care products, plant-based milk-/dairy-like food products, nut-based milk-/dairy-like food products). Some such alternatives may comprise isolated plant-derived components (e.g., purified or substantially purified carbohydrates, proteins, lipids, and vitamins). Increasingly also, alternatives to animal-derived food and other products are produced from specific components (e.g., proteins, lipids) that are produced recombinantly (e.g., using recombinant host cells).

The use of isolated components in food and other products poses new problems. One such problem is that food and other products produced from isolated components frequently contain a significant amount of such isolated components (e.g., more than was typical in previous products in which the components were utilized, and/or more than native components are comprised in corresponding animal-derived products). For example, a substitute dairy product that is produced using one or two isolated or recombinant milk proteins may comprise a higher content of that one or two isolated or recombinant milk proteins than a corresponding dairy product produced using bovine milk may comprise of the corresponding bovine milk protein (e.g., because bovine milk comprises a number of diverse milk proteins that fulfill functionalities in the dairy product that must all be provided for by the one or two isolated or recombinant milk proteins in the substitute dairy product). Moreover, food and other products produced from an isolated component may contain the isolated component in absence of other components that are typically comprised in the corresponding animal-derived products (e.g., an isolated or recombinant milk protein used for production of a dairy substitute product will be comprised in such dairy substitute product in absence of other milk proteins and/or milk lipids that are natively comprised in milk and in a corresponding dairy product). As a result, the isolated component may play a larger or different functional role in a food or other product than its corresponding native component may play in the corresponding animal-derived food or other product. When such larger or different functional role is a disadvantage, for example due to the isolated component having a property that negatively influences a property of the food or other product, methods must be developed for producing the food or other product using the isolated component that attenuate or eliminate such disadvantage.

Therefore, there exists a need for methods by which alternatives to animal-derived food and other products can be produced from isolated components, as well as for compositions obtained from such methods.

### INCORPORATION BY REFERENCE

All publications, patents, patent applications, sequences, database entries, scientific publications, and other references mentioned herein are incorporated by reference in their entireties to the same extent as if each individual publication, patent, patent application, sequence, database entry, scientific publication, or other reference was specifically and individually indicated to be incorporated by reference. To the extent the material incorporated by reference contradicts or is inconsistent with the present disclosure, the present disclosure, including definitions, will supersede any such material.

### SUMMARY OF THE INVENTION

In various aspects, provided herein is a method for producing a food product that comprises a substantial amount of a protein comprising a sulfur-comprising amino acid, wherein the method comprises: a) obtaining the protein (e.g., in substantially purified form); b) combining a substantial amount of the protein with a suitable amount of an oxidizing agent and optional one or more other ingredients to obtain a mixture; and c) subjecting the mixture to a process that results in production of the food product, wherein the process comprises exposing the sulfur-comprising amino acid.

The method of paragraph [0009], wherein the protein is a protein that was isolated from a natural source.

The method of paragraph [0009], wherein the protein is a recombinant protein.

The method of paragraph [0009], wherein the protein is a milk protein.

The method of paragraph [0009], wherein the protein is an isolated or recombinant β-lactoglobulin.

The method according to any of paragraphs [0009] through [0013], wherein the substantial amount is an amount that leads to production of an undesirable level of a volatile sulfur-containing compound.

The method according to any of paragraphs [0009] through [0014], wherein the protein is an isolated or recombinant β-lactoglobulin and the substantial amount is between 1% and 12% by mass of the isolated or recombinant β-lactoglobulin.

The method according to any of paragraphs [0009] through [0015], wherein the one or more other ingredients comprise a pH and/or ionic strength adjusting agent.

The method according to paragraph [0016], wherein the pH and/or ionic strength adjusting agent adjust pH of the mixture to between 6 and 8.

The method according to any of paragraphs [0009] through [0017], wherein the mixture further comprises more than 0.9% by mass of a lipid and an antioxidant.

The method according to any of paragraphs [0009] through [0018], wherein the oxidizing agents is selected from the group consisting of ascorbic acid, sodium ascorbate, azodicarbonamide, potassium bromate, potassium iodate, calcium iodate (lautarite), lipoxygenase, glucose oxidase, calcium peroxide, ammonium persulfate, potassium persulfate, ozone (e.g., sparged into the mixture), acetone peroxide, peracetic acid, chlorine, chlorine dioxide, benzoyl peroxide, rosemary extract, and hydrogen peroxide, dioxygen, and mixtures thereof.

The method according to paragraph [0019], wherein the oxidizing agents is a peroxide.

The method according to paragraph [0020], wherein the oxidizing agents is hydrogen peroxide.

The method according to any of paragraphs [0009] through [0021], wherein the protein is β-lactoglobulin, the oxidizing agents is hydrogen peroxide, and the suitable amount of the hydrogen peroxide is a molar ratio of less than 4 (e.g., less than 4, less than 3.5, less than 3, less than 2.5, less than 2, less than 1.5, or less than 1; between 0.5 and 3.8, 3.5, 3.2, 2.9, 2.6, 2.3, 2.0, 1.7, 1.4, 1.1, or 0.8; between 0.8 and 3.8, 3.5, 3.2, 2.9, 2.6, 2.3, 2.0, 1.7, 1.4, or 1.1; between 1.1 and 3.8, 3.5, 3.2, 2.9, 2.6, 2.3, 2.0, 1.7, or 1.4; between 1.4 and 3.8, 3.5, 3.2, 2.9, 2.6, 2.3, 2.0, or 1.7; between 1.7 and 3.8, 3.5, 3.2, 2.9, 2.6, 2.3, or 2.0; between 2.0 and 3.8, 3.5, 3.2, 2.9, 2.6, or 2.3; between 2.3 and 3.8, 3.5, 3.2, 2.9, or 2.6; between 2.6 and 3.8, 3.5, 3.2, or 2.9; between 2.9 and 3.8, 3.5, or 3.2; between 3.2 and 3.8, or 3.5; or between 3.5 and 3.8) of hydrogen peroxide to β-lactoglobulin.

The method according to any of paragraphs [0009] through [0022], wherein the exposing of the sulfur-comprising amino acid comprises high temperature heating.

The method according to any of paragraph [0024], wherein high temperature heating is heating at between 70° C. and 150° C. for between 1 second and 1 minute.

The method according to any of paragraphs [0009] through [0024], wherein the food product is a supplemented or substitute dairy product.

The method according to any of paragraphs [0009] through [0025], wherein the food product is food product is selected from the group consisting of a milk or barista beverage, a high protein ready-to-drink beverage, a frozen dessert mix, and a soft serve/shake mix/frozen dessert mix/creamer.

The method according to any of paragraphs [0009] through [0026], wherein the food product is essentially free of another a protein other than the protein comprising a sulfur-comprising amino acid.

The method according to any of paragraphs [0009] through [0026], wherein the food product is essentially free of a milk protein other than the protein comprising a sulfur-comprising amino acid.

### DETAILED DESCRIPTION OF THE INVENTION

The subsequent discussion of the invention is presented for purposes of illustration and description, and is not intended to limit the scope of the invention to the embodiments disclosed herein. As such, variations and modifications of the disclosed embodiments are within the scope of the invention, e.g., as may be within the skill and knowledge of those in the art after understanding the present disclosure. It is intended to obtain rights which include alternative embodiments to the extent permitted, including alternate, interchangeable and/or equivalent structures, functions, ranges or steps to those disclosed herein, and without intending to publicly dedicate any patentable subject matter. Unless defined otherwise, all technical and scientific terms used herein have the same meaning as is commonly understood by one of ordinary skill in the art to which this disclosure pertains. Further, unless otherwise required by context, singular terms shall include the plural, and plural terms shall include the singular.

### Definitions

The terms “a” and “an” and “the” and similar references as used herein refer to both the singular and the plural (e.g., meaning “at least one” or “one or more”), unless otherwise indicated herein or clearly contradicted by context. For example, the term “a compound” is synonymous with the terms “at least one compound” and “one or more compounds”, and may refer to a single compound or to a plurality of compounds, including mixtures thereof.

The term “and/or” as used herein refers to multiple components in combination with or exclusive of one another. For example, “x, y, and/or z” may refer to “x” alone, “y” alone, “z” alone, “x, y, and z”, “(x and y) or z”, “(x and z) or y”, “(y and z) or x”, “x and y” alone, “x and z” alone, “y and z” alone, or “x or y or z”.

The term “at least” or “one or more” as used herein refers to one, two, three, four, five, six, seven, eight, nine, ten, or more; at least one, at least two, at least three, at least four, at least five, at least six, at least seven, at least eight, at least nine, at least ten, or more; or all of the elements subsequently listed.

The term “casein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a casein natively found in a mammal-produced milk (i.e., a casein that is native to a mammal-produced milk; e.g., a native casein). Examples of caseins include β-casein, κ-casein, α-S1-casein, and α-S2-casein. Accordingly, the terms “β-casein”, “κ-casein”, “α-S1-casein”, and “α-S2-casein” as used herein refer to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a β-casein, κ-casein, α-S1-casein, and α-S2-casein, respectively, natively found in a mammal-produced milk (e.g., Bos taurus β-casein (amino acids 16 to 224 of UniProt sequence P02666), Bos taurus κ-casein (amino acids 22 to 190 of UniProt sequence P02668), Bos taurus α-S1-casein (amino acids 16 to 214 of UniProt sequence P02662), and Bos taurus α-S2-casein (amino acids 16 to 222 of UniProt sequence P02663), respectively). Differences between the amino acid sequences of a casein and that of a casein natively found in a mammal-produced milk may be due to conservative amino acid substitutions (i.e., replacement of amino acids with chemically similar amino acids; conservative substitution tables providing functionally similar amino acids are well known in the art), non-conservative amino acid substitutions, and amino acid insertions, amino acid deletions, and polypeptide truncations (e.g., providing a fragment of a casein) that do not materially alter structure and/or one or more functions of the casein. Such amino acid substitutions/deletions/insertions may be identified using methods known in the art, which involve producing a casein comprising a candidate amino acid substitution/deletion/insertion identified through molecular modeling approaches (using, for example, PyMol [Schrödinger, New York, NY]) or multi-sequence alignments (e.g., of orthologs of native caseins; using, for example, MUSCLE [Edgar, 2004, Nucleic Acids Res 32: 1792-1797]) as not materially altering protein structure and/or function, and testing such casein using well-known methods for determining protein structure and/or function. Such methods may also be employed to determine whether a casein is useful in the present invention, and to identify a casein that has an improved function in a specific application.

The term “disulfide bond” as used herein refers to a covalent bond between sulfur atoms that bind two polypeptides or different parts of one polypeptide (e.g., a covalent bond between sulfur atoms of two cysteine residue). For example, a protein that comprises an even number of cysteine residues may in non-reducing conditions comprise a disulfide bond.

The term “essentially free of” as used herein refers to the indicated component being either not detectable in the indicated composition by common analytical methods, or to the indicated component being present in such trace amount as to not be functional. The term “functional” as used in this context refers to not materially contributing to properties of the composition comprising the trace amount of the indicated component, or to not having material activity (e.g., chemical activity, enzymatic activity) in the indicated composition comprising the trace amount of the indicated component, or to not having health-adverse effects upon use or consumption of the composition comprising the trace amount of the indicated component. The term “materially contributing” as used herein refers to the indicated component contributing to an attribute of a composition to such extent that in the absence of the component (e.g., in a reference composition that is identical to the composition except that it lacks the indicated component) the attribute is at least 10%, at least 20%, at least 30%, at least 40%, or at least 50% less present/active/measurable.

The term “food product” as used herein refers to a composition that can be ingested by a human or an animal for dietary purposes (i.e., without ill health effects but with significant nutritional and/or caloric intake due to uptake of digested material in the gastrointestinal tract), including a domesticated animal (e.g., dog, cat), farm animal (e.g., cow, pig, horse), and wild animal (e.g., non-domesticated predatory animal). The term includes compositions that may be combined with or added to one or more other ingredients to make a food product that can be ingested by a human or an animal.

The term “thiol group” as used herein refers to the sidechain of a cysteine residue in which the hydrogen atom bound to the sulfur atom is not replaced by another atom (e.g., a —SH group of a cysteine residue that has not reacted with another —SH group to form a disulfide bond (—S—S—)). For example, a protein that comprises an odd number of cysteine residues may both in reducing and in non-reducing conditions comprise a thiol group.

The terms “including,” “includes,” “having,” “has,” “with,” or variants thereof as used herein are intended to be inclusive in a manner similar to the term “comprising”.

The term “mammal-produced milk” as used herein refers to a milk produced by a mammal.

The term “milk protein” as used herein refers to a whey protein or a casein. The milk protein may be derived from any mammalian species, including but not limited to cow, human, sheep, mouflon, goat, buffalo, camel, horse, donkey, alpaca, yak, llama, lemur, panda, guinea pig, squirrel, bear, macaque, gorilla, chimpanzee, mountain goat, monkey, ape, cat, dog, wallaby, rat, mouse, elephant, opossum, rabbit, whale, baboons, gibbons, orangutan, mandrill, pig, wolf, fox, lion, tiger, and echidna.

The term “native” as used herein refers to what is found in nature in its unmodified state (e.g., a cell that is not genetically modified by a human, and that is maintained under conditions [e.g., level of oxygenation, pH, salt concentration, temperature, and nutrient (e.g., carbon, nitrogen, sulfur) availability] that are not defined by a human).

The terms “optional” or “optionally” as used herein refer to a feature or structure being present or not, or an event or circumstance occurring or not. The description includes instances in which a feature or structure is present, instances in which a feature or structure is absent, instances in which an event or circumstance occurs, and instances in which an event or circumstance does not occur.

The term “oxidizing agent” as used herein refers to an agent that can oxidize a thiol group (e.g., a thiol group of a cysteine, a thiol group comprising in β-lactoglobulin).

The term “purifying” or “purified” or “isolating” or “isolated” as used herein refers to a component being substantially separated from chemicals (e.g., carbohydrates, lipids, ash, metabolites, signaling molecules, other proteins), cellular components (e.g., cell walls, membrane lipids, chromosomes), and cells (e.g., other cells in an organism) of the source from which the component originated. The component may be at least 60% pure, e.g., greater than 65%, 70%, 75%, 80%, 85%, 90%, 95%, or 99% pure. The term does not require (albeit allows) that the component be separated from all chemicals, cellular components, and cells. The term “recombinant” as used herein in reference to a protein (e.g., a milk protein) refers to a protein that is produced in a recombinant host cell, or to a protein that is synthesized from a recombinant polynucleotide.

The term “recombinant host cell” as used herein refers to a host cell that comprises a recombinant polynucleotide. Thus, for example, a recombinant host cell may produce a polynucleotide or polypeptide not found in the native (non-recombinant) form of the host cell, or a recombinant host cell may produce a polynucleotide or polypeptide at a level that is different from that in the native (non-recombinant) form of the host cell. It should be understood that such term is intended to refer not only to the particular subject cell but also to the progeny of such a cell. Because certain modifications may occur in succeeding generations due to either mutation or environmental influences, such progeny may not be identical to the subject cell, but are still included within the scope of the term “recombinant host cell” as used herein. A recombinant host cell may be an isolated cell or cell line grown in culture, or may be a cell which resides in a living tissue or organism.

The term “recombinant polynucleotide” as used herein refers to a polynucleotide that is removed from its naturally occurring environment, or a polynucleotide that is not associated with all or a portion of a polynucleotide abutting or proximal to the polynucleotide when it is found in nature, or a polynucleotide that is operatively linked to a polynucleotide that it is not linked to in nature, or a polynucleotide that does not occur in nature, or a polynucleotide that contains a modification that is not found in that polynucleotide in nature (e.g., insertion, deletion, or point mutation introduced artificially, e.g., by human intervention), or a polynucleotide that is integrated into a chromosome at a heterologous site. The term can be used, e.g., to describe cloned DNA isolates, or a polynucleotide comprising a chemically synthesized nucleotide analog. A polynucleotide is also considered “recombinant” if it contains a genetic modification that does not naturally occur. For instance, an endogenous polynucleotide is considered a “recombinant polynucleotide” if it contains an insertion, deletion, or substitution of one or more nucleotides that is introduced artificially (e.g., by human intervention). Such modification may introduce into the polynucleotide a point mutation, substitution mutation, deletion mutation, insertion mutation, missense mutation, frameshift mutation, duplication mutation, amplification mutation, translocation mutation, or inversion mutation. The term includes a polynucleotide in a host cell's chromosome, as well as a polynucleotide that is not in a host cell's chromosome (e.g., a polynucleotide that is comprised in an episome). A recombinant polynucleotide in a host cell or organism may replicate using the in vivo cellular machinery of the host cell; however, such recombinant polynucleotide, although subsequently replicated intracellularly, is still considered recombinant for purposes of this invention.

The term “sulfur-comprising” as used herein refers to comprising a sulfur atom. Accordingly, the term “sulfur-comprising amino acid” as used herein refers to an amino acid that comprises a sulfur atom. Non-limiting examples of sulfur-comprising amino acids include cysteine and methionine. Likewise, the term “sulfur-comprising compound” as used herein refers to a compound that comprises a sulfur atom. Non-limiting examples of sulfur-comprising compounds include hydrogen sulfide, dimethyl sulfide, and dimethyl disulfide.

The term “whey protein” as used herein refers to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in a whey protein natively found in a mammal-produced milk (i.e., a whey protein that is native to a mammal-produced milk; e.g., a native whey protein). Examples of whey proteins include α-lactalbumin, β-lactoglobulin, lactotransferrin, lactoferricin, serum albumin protein, lactoperoxidase protein, and glycomacropeptide. Accordingly, the terms “α-lactalbumin”, “β-lactoglobulin”, “lactotransferrin”, “lactoferricin”, “serum albumin”, “lactoperoxidase”, and “glycomacropeptide” as used herein refer to a polypeptide that comprises a sequence of at least 20 (e.g., at least 20, at least 30, at least 40, at least 50, at least 60, at least 70, at least 80, at least 90, at least 100, at least 150) amino acids that is at least 40% (e.g., at least 40%, at least 45%, at least 50%, at least 55%, at least 60%, at least 65%, at least 70%, at least 75%, at least 80%, at least 85%, at least 90%, at least 95%, at least 99%, 100%) identical to a sequence of amino acids in an α-lactalbumin, β-lactoglobulin, lactotransferrin, lactoferricin, serum albumin, lactoperoxidase, and glycomacropeptide (GMP), respectively, natively found in a mammal-produced milk (e.g., Bos taurus α-lactalbumin (amino acids 20-142 of UniProt sequence P00711), Bos taurus β-lactoglobulin (amino acids 17-178 of UniProt sequence P02754), Bos taurus lactotransferrin (amino acids 20 to 708 of UniProt sequence P24627), Bos taurus lactoferricin (amino acids 36 to 60 of UniProt sequence P24627), Bos taurus serum albumin (amino acids 25 to 607 of UniProt sequence P02769), Bos taurus lactoperoxidase (amino acids 101 to 712 of UniProt sequence P80025), and Bos taurus glycomacropeptide (GMP; amino acids 127 to 190 of UniProt sequence P02668), respectively). Differences between the amino acid sequences of a whey protein and that of a whey protein natively found in a mammal-produced milk may be due to conservative amino acid substitutions (i.e., replacement of amino acids with chemically similar amino acids; conservative substitution tables providing functionally similar amino acids are well known in the art), non-conservative amino acid substitutions, amino acid insertions, amino acid deletions, and polypeptide truncations (e.g., providing a fragment of a whey protein) that do not materially alter structure and/or one or more functions of the whey protein. Such amino acid substitutions/deletions/insertions may be identified using methods known in the art, which involve producing a whey protein comprising a candidate amino acid substitution/deletion/insertion identified through molecular modeling approaches (using, for example, PyMol [Schrödinger, New York, NY]) or multi-sequence alignments (e.g., of orthologs of native whey proteins; using, for example, MUSCLE [Edgar, 2004, Nucleic Acids Res 32: 1792-1797]) as not materially altering protein structure and/or function, and testing such whey protein using well-known methods for determining protein structure and/or function. Such methods may also be employed to determine whether a whey protein is useful in the present invention, and to identify a whey protein that has an improved function in a specific application.

The term “% by mass” as used herein refers to a percentage value for a mass as determined in a hydrated composition, such that the composition includes the mass of powder as well as the mass of the hydrating agent, with 100% fixed as the percentage value for the entire hydrated composition. In embodiments in which the composition is in powder form to which the mass of the hydrating agent will be added at a later time, the term refers to a percentage value for a mass as determined relative to the eventual entire hydrated composition (with 100% fixed as the percentage value for that entire eventual hydrated composition).

Recitation of ranges of values herein are merely intended to serve as a shorthand method of referring individually to each separate value (fractional or integral) falling within the range inclusive of the recited minimum and maximum value, unless otherwise indicated herein, and each separate value is incorporated into the specification as if it were individually recited herein. Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of less than or equal to 10. It should further be understood that all ranges and quantities described below are approximations and are not intended to limit the invention.

It should be understood that in any method disclosed herein the order of steps or order for performing certain actions is immaterial so long as the invention remains operable. Moreover, two or more steps or actions may be conducted simultaneously.

### Method for Producing Food Product Comprising Protein Comprising Sulfur-Comprising Amino Acid

In various aspects, provided herein is a method for producing a food product that comprises a substantial amount of a protein comprising a sulfur-comprising amino acid, wherein the method comprises: a) obtaining the protein (e.g., in substantially purified form); b) combining a substantial amount of the protein with a suitable amount of an oxidizing agent and optional one or more other ingredients to obtain a mixture; and c) subjecting the mixture to a process that results in production of the food product, wherein the process comprises exposing the sulfur-comprising amino acid (i.e., exposing such that the sulfur-comprising amino acid is no longer buried within the protein and shielded from interaction with components in the environment [e.g., shielded from interacting with the oxidizing agent]).

Without wishing to be bound by theory, a protein comprising a sulfur-comprising amino acid when subjected to conditions that expose the sulfur-comprising amino acid may release volatile sulfur-comprising compounds that may cause sulfury flavor, taste, and/or aroma notes. Release of volatile sulfur-comprising compounds from proteins present at low levels in a food product and/or in combination with other proteins (as is the case, for example, for milk proteins comprised in dairy products) may be desirable as the volatile sulfur-comprising compounds may contribute to a desired taste, flavor, and/or aroma profile. However, when a protein comprising a sulfur-comprising amino acid is used at elevated levels (e.g., to impart a desired characteristic and/or functionality in a food product), as is the case, for example, in production of food products using one to a small number of purified proteins or recombinant proteins, release of volatile sulfur-comprising compounds can lead to noticeable flavor, taste, and/or aroma notes that can make the food products unpalatable. Addition of an oxidizing agent during preparation of the food product may prevent release of such volatile sulfur-comprising compounds, and/or oxidize such volatile sulfur-comprising compounds to non-volatile compounds (e.g., an oxidizing agent may convert a thiol group of a cysteine comprised in a protein into a cysteine sulphonic acid and/or cysteic acid, and thereby remove the potential for release of a volatile sulfur-comprising compound). Therefore, in the method provided herein, addition of an oxidizing agent to the mixture permits production of a food product that does not produce substantial (e.g., detectable by a sensory expert) levels of flavor, taste, and/or aroma notes caused by the presence of a substantial level of a protein comprising a sulfur-comprising amino acid.

### Protein Comprising Sulfur-Comprising Amino Acid

The protein comprised at substantial level in a food product produced by a method according to the above may be any protein comprising a sulfur-comprising amino acid.

The protein may a protein that was isolated from a natural source. For example, the protein may be a milk protein that was isolated from milk, or a plant protein that was isolated from a plant. Methods for isolating protein from natural sources are known and used to produce protein isolates, protein concentrates, protein flours, and partially purified or purified proteins. Alternatively, the protein may be a recombinant protein. For example, the protein may be a recombinant milk protein (e.g., a recombinant casein or a recombinant whey protein). Non-limiting examples of milk proteins comprising a sulfur-comprising amino acid include β-lactoglobulin (e.g., Bos taurus β-lactoglobulin [amino acids 17-178 of UniProt sequence P02754]), serum albumin (e.g., Bos taurus serum albumin protein [amino acids 25 to 607 of UniProt sequence P02769]), lactoperoxidase (e.g., Bos taurus lactoperoxidase [amino acids 101 to 712 of UniProt sequence P80025]), α-lactalbumin (e.g., Bos taurus α-lactalbumin [amino acids 20-142 of UniProt sequence P00711]), lactotransferrin (e.g., Bos taurus lactotransferrin [amino acids 20 to 708 of UniProt sequence P24627]), lactoferricin (e.g., Bos taurus lactoferricin [amino acids 36 to 60 of UniProt sequence P24627]), glycomacropeptide (GMP; e.g., Bos taurus GMP [amino acids 127 to 190 of UniProt sequence P02668]), β-casein (e.g., Bos taurus β-casein [amino acids 16 to 224 of UniProt sequence P02666]), κ-casein (e.g., Bos taurus κ-casein [amino acids 22 to 190 of UniProt sequence P02668]), α-S1-casein (e.g., Bos taurus α-S1-casein [amino acids 16 to 214 of UniProt sequence P02662]), and α-S2-casein (e.g., Bos taurus α-S2-casein [amino acids 16 to 222 of UniProt sequence P02663]). Further examples of proteins comprising a sulfur-comprising amino acid include structural animal proteins (e.g., collagen, tropoelastin, elastin), egg proteins (e.g., hereinovomucoid, ovalbumin, ovotransferrin, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y), and globular proteins (e.g., albumin).

Those of skill in the art can readily ascertain whether a protein comprises a sulfur-comprising amino acid (e.g., by determining whether a protein comprises a cysteine or methionine residue, and/or by analyzing the structure of a protein with regard to the presence of a sulfur-comprising amino acid using methods known in the art, such as, for example, mass spectrometry (MS) analysis following proteolytic digestion of a protein under partial reducing or non-reducing conditions, and analysis by mass of protein crystal structures).

Methods for obtaining a recombinant protein are known in the art. Methods for obtaining a recombinant milk protein comprising a sulfur-comprising amino acid (e.g., a recombinant β-lactoglobulin) are disclosed, for example, in PCT filing PCT/US2015/046428 filed Aug. 21, 2015, PCT filing PCT/US2017/48730 filed Aug. 25, 2017, and PCT/US2020/029392 filed Apr. 22, 2020. The recombinant protein may be produced using a recombinant host cell derived from any organism, including any bacterium, fungus (e.g., yeast, filamentous fungus), archaea, protista, animal (including any unicellular animal), plant (including any unicellular plant), algae, protozoan, and chromista, or from a genetic variant (e.g., mutant) thereof, as well as from any generally recognized as safe (GRAS) industrial host cell. The recombinant protein may be purified (e.g., purified from the source from which the recombinant protein originated [e.g., from the recombinant host cell that was used for producing the recombinant protein]). For example, the recombinant protein may be at least 30%, at least 40%, at least 50%, at least 60%, at least 65%, at least 70%, at least 75%, at least 80%, at least 85%, at least 90%, at least 95%, at least 98%, or 100% pure.

The substantial amount of the protein comprised in a food product produced by a method according to any of the above may be an amount of the protein that when used in the production of a food product by a method according to any of the above leads to production of an undesirable level of a volatile sulfur-containing compound (e.g., a level of the volatile sulfur-containing compound that confers an undesirable taste, flavor, and/or aroma note on the food product, and/or makes the food product unpalatable). A substantial amount of the protein may be, for example, at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, less than 12%, less than 11%, less than 10%, less than 9%, less than 8%, less than 7%, less than 6%, less than 5%, less than 4%, less than 3%, less than 2%; between 1% and 12%, 11%, 10%, 9%, 8%, 7%, 6%, 5%, 4%, 3%, or 2%; between 2% and 12%, 11%, 10%, 9%, 8%, 7%, 6%, 5%, 4%, or 3%; between 3% and 12%, 11%, 10%, 9%, 8%, 7%, 6%, 5%, or 4%; between 4% and 12%, 11%, 10%, 9%, 8%, 7%, 6%, or 5%; between 5% and 12%, 11%, 10%, 9%, 8%, 7%, or 6%; between 6% and 12%, 11%, 10%, 9%, 8%, or 7%; between 7% and 12%, 11%, 10%, 9%, or 8%; between 8% and 12%, 11%, 10%, or 9%; between 9% and 12%, 11%, or 10%; between 10% and 12%, or 11%; or between 11% and 12% by mass of the protein comprised in the food product. Production of an undesirable level of a volatile sulfur-containing compound may be determined by analyzing headspace by GC/MS (as thresholds for perception of volatile sulfur-containing compounds are known in the art), or by evaluation by trained sensory experts.

### Oxidizing Agent

The oxidizing agent used in a method according to any of the above may be any oxidizing agent suitable for use in production of a food product. For example, the oxidizing agent may be certified to be safe for use in a food product (e.g., generally recognized as safe [GRAS] certified).

Non-limiting examples of oxidizing agents include ascorbic acid, sodium ascorbate, azodicarbonamide, potassium bromate, potassium iodate, calcium iodate (lautarite), lipoxygenase, glucose oxidase, calcium peroxide, ammonium persulfate, potassium persulfate, ozone (e.g., sparged into the mixture), acetone peroxide, peracetic acid, chlorine, chlorine dioxide, benzoyl peroxide, rosemary extract, and hydrogen peroxide, dioxygen, and mixtures thereof.

The suitable amount of the oxidizing agent used in a method according to any of the above may be, for example, between 35 ppm and 75 ppm, 70 ppm, 65 ppm, 60 ppm, 55 ppm, 50 ppm, 45 ppm, or 40 ppm; between 40 ppm and 75 ppm, 70 ppm, 65 ppm, 60 ppm, 55 ppm, 50 ppm, or 45 ppm; between 45 ppm and 75 ppm, 70 ppm, 65 ppm, 60 ppm, 55 ppm, or 50 ppm; between 50 ppm and 75 ppm, 70 ppm, 65 ppm, 60 ppm, or 55 ppm; between 55 ppm and 75 ppm, 70 ppm, 65 ppm, or 60 ppm; between 60 ppm and 75 ppm, 70 ppm, or 65 ppm; between 65 ppm and 75 ppm, or 70 ppm; or between 70 ppm and 75 ppm of hydrogen peroxide per between 3% and 7%, 6.5%, 6%, 5.5%, 5%, 4.5%, 4%, or 3.5%; between 3.5% and 7%, 6.5%, 6%, 5.5%, 5%, 4.5%, or 4%; between 4% and 7%, 6.5%, 6%, 5.5%, 5%, or 4.5%; between 4.5% and 7%, 6.5%, 6%, 5.5%, or 5%; between 5% and 7%, 6.5%, 6%, or 5.5%; between 5.5% and 7%, 6.5%, or 6%; between 6% and 7%, or 6.5%; or between 6.5% and 7% by mass of protein comprising a sulfur-comprising amino acid residue (e.g., a β-lactoglobulin [e.g., a recombinant β-lactoglobulin]) comprised in the mixture.

The suitable amount of the oxidizing agent used in a method according to any of the above may be, for example, between 1 mM and 3 mM, 2.75 mM, 2.5 mM, 2.25 mM, 2 mM, 1.75 mM, 1.5 mM, or 1.25 mM; between 1.25 mM and 3 mM, 2.75 mM, 2.5 mM, 2.25 mM, 2 mM, 1.75 mM, or 1.5 mM; between 1.5 mM and 3 mM, 2.75 mM, 2.5 mM, 2.25 mM, 2 mM, or 1.75 mM; between 1.75 mM and 3 mM, 2.75 mM, 2.5 mM, 2.25 mM, or 2 mM; between 2 mM and 3 mM, 2.75 mM, 2.5 mM, or 2.25 mM; between 2.25 mM and 3 mM, 2.75 mM, or 2.5 mM; between 2.5 mM and 3 mM, or 2.75 mM; or between 2.75 mM and 3 mM of hydrogen peroxide per between 1 mM and 4 mM, 3.75 mM, 3.5 mM, 3.25 mM, 3 mM, 2.75 mM, 2.5 mM, 2.25 mM, 2 mM, 1.75 mM, 1.5 mM, or 1.25 mM; between 1.25 mM and 4 mM, 3.75 mM, 3.5 mM, 3.25 mM, 3 mM, 2.75 mM, 2.5 mM, 2.25 mM, 2 mM, 1.75 mM, or 1.5 mM; between 1.5 mM and 4 mM, 3.75 mM, 3.5 mM, 3.25 mM, 3 mM, 2.75 mM, 2.5 mM, 2.25 mM, 2 mM, or 1.75 mM; between 1.75 mM and 4 mM, 3.75 mM, 3.5 mM, 3.25 mM, 3 mM, 2.75 mM, 2.5 mM, 2.25 mM, or 2 mM; between 2 mM and 4 mM, 3.75 mM, 3.5 mM, 3.25 mM, 3 mM, 2.75 mM, 2.5 mM, or 2.25 mM; between 2.25 mM and 4 mM, 3.75 mM, 3.5 mM, 3.25 mM, 3 mM, 2.75 mM, or 2.5 mM; between 2.5 mM and 4 mM, 3.75 mM, 3.5 mM, 3.25 mM, 3 mM, or 2.75 mM; between 2.75 mM and 4 mM, 3.75 mM, 3.5 mM, 3.25 mM, or 3 mM; between 3 mM and 4 mM, 3.75 mM, 3.5 mM, or 3.25 mM; between 3.25 mM and 4 mM, 3.75 mM, or 3.5 mM; between 3.5 mM and 4 mM, or 3.75 mM; or between 3.75 mM and 4 mM of protein comprising a sulfur-comprising amino acid residue (e.g., β-lactoglobulin [e.g., recombinant β-lactoglobulin]) comprised in the mixture.

The suitable amount of the oxidizing agent used in a method according to any of the above may be, for example, between 1 mM and 3 mM, 2.75 mM, 2.5 mM, 2.25 mM, 2 mM, 1.75 mM, 1.5 mM, or 1.25 mM; between 1.25 mM and 3 mM, 2.75 mM, 2.5 mM, 2.25 mM, 2 mM, 1.75 mM, or 1.5 mM; between 1.5 mM and 3 mM, 2.75 mM, 2.5 mM, 2.25 mM, 2 mM, or 1.75 mM; between 1.75 mM and 3 mM, 2.75 mM, 2.5 mM, 2.25 mM, or 2 mM; between 2 mM and 3 mM, 2.75 mM, 2.5 mM, or 2.25 mM; between 2.25 mM and 3 mM, 2.75 mM, or 2.5 mM; between 2.5 mM and 3 mM, or 2.75 mM; or between 2.75 mM and 3 mM of hydrogen peroxide per between 1 mM and 4 mM, 3.75 mM, 3.5 mM, 3.25 mM, 3 mM, 2.75 mM, 2.5 mM, 2.25 mM, 2 mM, 1.75 mM, 1.5 mM, or 1.25 mM; between 1.25 mM and 4 mM, 3.75 mM, 3.5 mM, 3.25 mM, 3 mM, 2.75 mM, 2.5 mM, 2.25 mM, 2 mM, 1.75 mM, or 1.5 mM; between 1.5 mM and 4 mM, 3.75 mM, 3.5 mM, 3.25 mM, 3 mM, 2.75 mM, 2.5 mM, 2.25 mM, 2 mM, or 1.75 mM; between 1.75 mM and 4 mM, 3.75 mM, 3.5 mM, 3.25 mM, 3 mM, 2.75 mM, 2.5 mM, 2.25 mM, or 2 mM; between 2 mM and 4 mM, 3.75 mM, 3.5 mM, 3.25 mM, 3 mM, 2.75 mM, 2.5 mM, or 2.25 mM; between 2.25 mM and 4 mM, 3.75 mM, 3.5 mM, 3.25 mM, 3 mM, 2.75 mM, or 2.5 mM; between 2.5 mM and 4 mM, 3.75 mM, 3.5 mM, 3.25 mM, 3 mM, or 2.75 mM; between 2.75 mM and 4 mM, 3.75 mM, 3.5 mM, 3.25 mM, or 3 mM; between 3 mM and 4 mM, 3.75 mM, 3.5 mM, or 3.25 mM; between 3.25 mM and 4 mM, 3.75 mM, or 3.5 mM; between 3.5 mM and 4 mM, or 3.75 mM; or between 3.75 mM and 4 mM of thiol groups comprised in the mixture.

The suitable amount of the oxidizing agent used in a method according to any of the above may be, for example, between 1 mM and 3 mM, 2.75 mM, 2.5 mM, 2.25 mM, 2 mM, 1.75 mM, 1.5 mM, or 1.25 mM; between 1.25 mM and 3 mM, 2.75 mM, 2.5 mM, 2.25 mM, 2 mM, 1.75 mM, or 1.5 mM; between 1.5 mM and 3 mM, 2.75 mM, 2.5 mM, 2.25 mM, 2 mM, or 1.75 mM; between 1.75 mM and 3 mM, 2.75 mM, 2.5 mM, 2.25 mM, or 2 mM; between 2 mM and 3 mM, 2.75 mM, 2.5 mM, or 2.25 mM; between 2.25 mM and 3 mM, 2.75 mM, or 2.5 mM; between 2.5 mM and 3 mM, or 2.75 mM; or between 2.75 mM and 3 mM of hydrogen peroxide per between 5 mM and 20 mM, 18.75 mM, 17.5 mM, 16.25 mM, 15 mM, 13.75 mM, 12.5 mM, 11.25 mM, 10 mM, 8.75 mM, 7.5 mM, or 6.25 mM; per between 6.25 mM and 20 mM, 18.75 mM, 17.5 mM, 16.25 mM, 15 mM, 13.75 mM, 12.5 mM, 11.25 mM, 10 mM, 8.75 mM, or 7.5 mM; per between 7.5 mM and 20 mM, 18.75 mM, 17.5 mM, 16.25 mM, 15 mM, 13.75 mM, 12.5 mM, 11.25 mM, 10 mM, or 8.75 mM; per between 8.75 mM and 20 mM, 18.75 mM, 17.5 mM, 16.25 mM, 15 mM, 13.75 mM, 12.5 mM, 11.25 mM, or 10 mM; per between 10 mM and 20 mM, 18.75 mM, 17.5 mM, 16.25 mM, 15 mM, 13.75 mM, 12.5 mM, or 11.25 mM; per between 11.25 mM and 20 mM, 18.75 mM, 17.5 mM, 16.25 mM, 15 mM, 13.75 mM, or 12.5 mM; per between 12.5 mM and 20 mM, 18.75 mM, 17.5 mM, 16.25 mM, 15 mM, or 13.75 mM; per between 13.75 mM and 20 mM, 18.75 mM, 17.5 mM, 16.25 mM, or 15 mM; per between 15 mM and 20 mM, 18.75 mM, 17.5 mM, or 16.25 mM; per between 16.25 mM and 20 mM, 18.75 mM, or 17.5 mM; per between 17.5 mM and 20 mM, or 18.75 mM; or per between 18.75 mM and 20 mM of amino acids comprising a sulfur atom comprised in the mixture.

The suitable amount of the oxidizing agent used in a method according to any of the above may be, for example, a molar ratio of less than 4, less than 3.5, less than 3, less than 2.5, less than 2, less than 1.5, or less than 1; between 0.5 and 3.8, 3.5, 3.2, 2.9, 2.6, 2.3, 2.0, 1.7, 1.4, 1.1, or 0.8; between 0.8 and 3.8, 3.5, 3.2, 2.9, 2.6, 2.3, 2.0, 1.7, 1.4, or 1.1; between 1.1 and 3.8, 3.5, 3.2, 2.9, 2.6, 2.3, 2.0, 1.7, or 1.4; between 1.4 and 3.8, 3.5, 3.2, 2.9, 2.6, 2.3, 2.0, or 1.7; between 1.7 and 3.8, 3.5, 3.2, 2.9, 2.6, 2.3, or 2.0; between 2.0 and 3.8, 3.5, 3.2, 2.9, 2.6, or 2.3; between 2.3 and 3.8, 3.5, 3.2, 2.9, or 2.6; between 2.6 and 3.8, 3.5, 3.2, or 2.9; between 2.9 and 3.8, 3.5, or 3.2; between 3.2 and 3.8, or 3.5; or between 3.5 and 3.8 of oxidizing agent (e.g., hydrogen peroxide) to sulfur-comprising protein (e.g., β-lactoglobulin [e.g., recombinant β-lactoglobulin]) comprised in the mixture.

The suitable amount of the oxidizing agent may be added directly to the mixture in a method according to any of the above, or may be generated in the mixture (e.g., generated via use of an enzyme capable of producing the oxidizing agent [e.g., lactose oxidase, hexose oxidase], electrochemically generated).

### Exposing Sulfur-Comprising Amino Acid

The exposing of the sulfur-comprising amino acid comprised in the protein in a method according to any of the above may occur by any means known in the art and used for the production of a food product. Non-limiting examples of means include high temperature heating (e.g., pasteurizing, ultra-pasteurizing), high pressure processing (e.g., exposure to a pressure of between 20 bar and 4000 bar), mechanical pressure processing (e.g., shearing), extruding (e.g., high-moisture extruding, low-moisture extruding), exposure to specific pH, and combinations thereof.

Exposing of the sulfur-comprising amino acid comprised in a protein in a method according to any of the above via heating at high temperature provides advantages due to simplicity of the process and ability to easily incorporate the denaturing in a food production process (e.g., in-line incorporation in a batch process). It further provides ideal conditions for preventing over-oxidation of an ingredient comprised in the mixture (e.g., the protein comprising a sulfur-comprising amino acid, which may lead to protein aggregation and/or undesired oxidation of other amino acids comprised in the protein), particularly when low levels of the oxidizing agent are combined with denaturation via heating at high temperature, as the high temperature aside from aiding in denaturation may also serve to remove unreacted residual oxidizing agent (e.g., dissipate excess hydrogen peroxide) and terminate the oxidation reaction without need for an active termination step (e.g., without requiring addition of an enzyme [e.g., catalase capable of dismutating a peroxide], catalysator, or reactant [e.g., antioxidant] that promotes elimination of residual oxidizing agent).

The process for producing a food product by a method according to any of the above may further comprise any one or any combination of the following: homogenizing, adding a microbial culture (e.g., a thermophilic culture, a mesophilic culture), fermenting (e.g., to produce a fermented dairy product [e.g., yogurt, cream cheese, sour cream]), adjusting pH, spray drying, and additional heating steps.

### Other Ingredients

The one or more other ingredients comprised in a food product produced by a method according to any of the above may be any suitable other ingredient. Non-limiting examples of suitable other ingredients include proteins (e.g., plant proteins [e.g., pea proteins, soy proteins, potato proteins, rice proteins]), bioactive agents (e.g., neutraceuticals), nutritional agents (e.g., nutritional supplements, pro-vitamins, vitamins, minerals, antioxidants, essential and semi-essential amino acids), functional agents (e.g., acidulants, buffering agents, shelf life extending agents, pH and/or ionic strength adjusting agents, anti-microbial agents, preservatives, emulsifiers, plasticizers, texturing/mouthfeel agents, coloring agents, taste/flavor agents, aroma agents, leavening agents, flow agents), carbohydrates (e.g., monosaccharides, disaccharides, polysaccharides [e.g., starches, flours, gums, edible fibers]), lipids (e.g., fats, oils, monoglycerides, diglycerides, triglycerides, phospholipids, free fatty acids) and sweetening agents (e.g., protein-based sweetening agents [e.g., brazzein (UniProt sequence P56552), curculin (UniProt sequence P19667 amino acids 23 to 136, Q6F495 amino acids 23 to 135, respectively), mabinlin (UniProt sequences P80351 amino acids 1 to 32, P80351 amino acids 33 to 104, P30233 amino acids 36 to 68, P30233 amino acids 83 to 154, P80352 amino acids 1 to 32, P80352 amino acids 33 to 104, P80353 amino acids 1 to 28, P80353 amino acids 29 to 100), miraculin (UniProt sequence P13087 amino acids 30 to 220), monelin (UniProt sequences P02881, P02882), pentadin, thaumatin (UniProt sequences P02883 amino acids 23 to 229, P02884 amino acids 23 to 229)]). The one or more other ingredients may not be derived from an animal, but rather may be derived from a plant.

When the food product produced by a method according to any comprises a significant amount of lipids (e.g., more than 0.9% by mass) addition of an antioxidant (e.g., tocopherol) to the mixture is advantageous to prevent over-oxidation of the lipids and production of other undesired tastes, flavors, and/or aromas.

### Food Product

The food product produced by a method according to any of the above may be any food product that can be produced by a process that comprises exposing a sulfur-comprising amino acid comprised in a protein comprised in the food product.

The food product may be selected from any of the food product categories defined by the National Health and Nutrition Examination Survey (NHANES). Non-limiting examples of NHANES food product categories include snack foods and gums (e.g., snack bars, crackers, salty snacks from grain products, chewing gums); breads, grains, and pastas (e.g., oat breads and rolls, cornbread, corn muffins, tortillas, flour and dry mixes, biscuits, multi-grain breads and rolls, whole wheat breads and rolls, pastas, rye breads and rolls, cracked wheat breads and rolls, white breads and rolls, bagels, sweet buns, loaf breads, baguettes, pizza crusts, breadsticks, brioches, challahs, biscuits, hot dog rolls, hamburger buns, crispbreads, flatbreads, wraps, biscuits, gluten-free breads, pretzels, crackers, donuts, beignets, liege waffles); beverages (e.g., beers and ales, beverage concentrates, beverages, energy drinks, sports drinks, fluid replacements, soft drinks, carbonated beverages, juices, wines, beers, cocktails, nutrition drinks, nutrition powders, protein-enriched beverages, coffee, tea); sweets and desserts (e.g., cakes [e.g., pound cake, yellow cake, devil's food cake, angel food cake, sponge cake, chiffon cake, tube cake, rolled cake, oil cake, castellas, pancakes, muffins, cupcakes, brownies, madeleines, cornbread, flourless cakes, molten chocolate cakes, financier, dacquoise, cheesecake, shortcake], candies, chips, cookies [biscotti, chocolate chip cookies, sugar cookies, waffle cookies, tuile, macaron, coconut macaroons], cobblers, pastries [e.g., danish, choux, pie crusts, puff pastry, croissants, crepes, tarts, scones, meringue, waffles, canelé, pate sucree, sable, souffles, marshmellows], ices or popsicles, muffins, pies, sugar replacements or substitutes, syrups, honey, jellies, jams, preserves, salads, crepes, Danish, breakfast pastries, doughnuts); breakfast foods (e.g., cereal grains, cereal, rice, French toast, pancakes, waffles, coffee cake); salad dressings, oils, sauces, condiments (e.g., cooking fats, vegetable oils, salad dressings, tomato sauces, gravies); potatoes (e.g., potato salad, potato soups, chips and sticks, fried potatoes, mashed potatoes, stuffed potatoes, puffs); and soups (e.g., vegetable soups, vegetable broths), meals, main dishes, proteins (e.g., meat substitutes), and seafoods.

The food product may be a supplemented food product (i.e., a conventional food product that is supplemented with a protein comprising a sulfur-comprising amino acid), or may be a substitute food product (i.e., a food product that comprises a sulfur-comprising amino acid and that resembles a conventional food product and/or that can be used in place of a conventional food product).

The food product may be a supplemented dairy product (i.e., a conventional dairy product that is supplemented with a protein comprising a sulfur-comprising amino acid) or a substitute dairy product (i.e., a food product that comprises a sulfur-comprising amino acid and that resembles a conventional dairy product and/or that can be used in place of a conventional food product). The term “dairy product” as used herein refers to milk (e.g., whole milk [at least 3.25% milk fat], partly skimmed milk [from 1% to 2% milk fat], skim milk [less than 0.2% milk fat], cooking milk, condensed milk, flavored milk, goat milk, sheep milk, dried milk, evaporated milk, milk foam), and products derived from milk, including but not limited to yogurt (e.g., whole milk yogurt [at least 6 grams of fat per 170 g], low-fat yogurt [between 2 and 5 grams of fat per 170 g], nonfat yogurt [0.5 grams or less of fat per 170 g], greek yogurt [strained yogurt with whey removed], whipped yogurt, goat milk yogurt, Labneh [labne], sheep milk yogurt, yogurt drinks [e.g., whole milk Kefir, low-fat milk Kefir], Lassi), cheese (e.g., whey cheese such as ricotta; pasta filata cheese such as mozzarella; semi-soft cheese such as Havarti and Muenster; medium-hard cheese such as Swiss and Jarlsberg and halloumi; hard cheese such as Cheddar and Parmesan; washed curd cheese such as Colby and Monterey Jack; soft ripened cheese such as Brie and Camembert; fresh cheese such as cottage cheese, feta cheese, cream cheese, paneer, and curd), processed cheese, processed cheese food, processed cheese product, processed cheese spread, enzyme-modulated cheese; cold-pack cheese), dairy-based sauces (e.g., salad dressing, bechamel sauce, fresh sauces, frozen sauces, refrigerated sauces, shelf stable sauces), dairy spreads (e.g., low-fat spread, low-fat butter), cream (e.g., dry cream, heavy cream, light cream, whipping cream, half-and-half, coffee whitener, coffee creamer, sour cream, creme fraiche), frozen confections (e.g., ice cream, smoothie, milk shake, frozen yogurt, sundae, gelato, custard), dairy desserts (e.g., fresh, refrigerated, or frozen), butter (e.g., whipped butter, cultured butter), dairy powders (e.g., whole milk powder, skim milk powder, fat-filled milk powder (i.e., milk powder comprising plant fat in place of all or some animal fat), infant formula, milk protein concentrate (e.g., milk protein concentrate, whey protein concentrate, demineralized whey protein concentrate, β-lactoglobulin concentrate, α-lactalbumin concentrate, GMP concentrate, casein concentrate), milk protein isolate (e.g., milk protein isolate, whey protein isolate, demineralized whey protein isolate, β-lactoglobulin protein isolate, α-lactalbumin protein isolate, GMP protein isolate, casein protein isolate), nutritional supplements, texturizing blends, flavoring blends, coloring blends, ready-to-drink or ready-to-mix products (e.g., fresh, refrigerated, or shelf stable dairy protein beverages, weight loss beverages, nutritional beverages, sports recovery beverages, and energy drinks), puddings, gels, chewables, crisps, bars (e.g., nutrition bars, protein bars), and fermented dairy products (e.g., yoghurt, cheese, sour cream, cultured buttermilk, cultured butter, cultured butter oil).

The food product may be a supplemented animal meat product (i.e., a conventional animal meat product that is supplemented with a protein comprising a sulfur-comprising amino acid) or a substitute animal meat or animal meat product (i.e., a food product that comprises a sulfur-comprising amino acid and that resembles a conventional animal meat or animal meat product and/or that can be used in place of a conventional animal meat or animal meat product). Non-limiting examples of animal meats and animal meat products include flesh obtained from skeletal muscle or from other organs (e.g., kidney, heart, liver, gallbladder, intestine, stomach, bone marrow, brain, thymus, lung, tongue), or parts thereof, obtained from an animal. The animal meat may be dark or white meat. Non-limiting examples of animals from which animal meat or animal meat product can be obtained include cattle, lamb, mutton, horse, poultry (e.g., chicken, duck, goose, turkey), fowl (e.g., pigeon, dove, grouse, partridge, ostrich, emu, pheasant, quail), fresh or salt water fish (e.g., catfish, tuna, spearfish, shark, halibut, sturgeon, salmon, bass, muskie, pike, bowfin, gar, eel, paddlefish, bream, carp, trout, walleye, snakehead, crappie, sister, mussel, scallop, abalone, squid, octopus, sea urchin, cuttlefish, tunicate), crustacean (e.g., crab, lobster, shrimp, barnacle), game animal (e.g., deer, fox, wild pig, elk, moose, reindeer, caribou, antelope, zebra, squirrel, marmot, rabbit, bear, beaver, muskrat, opossum, raccoon, armadillo, porcupine, bison, buffalo, boar, lynx, bobcat, bat), reptile (e.g., snakes, turtles, lizards, alligators, crocodiles), any insect or other arthropod, rodent (nutria, guinea pig, rat, mice, vole, groundhog, capybara), kangaroo, whale, and seal. The animal meat or animal meat product may be ground, chopped, shredded, or otherwise processed, and uncooked, cooking, or cooked.

The food product may be a supplemented egg product (i.e., a conventional egg product that is supplemented with a protein comprising a sulfur-comprising amino acid) or a substitute egg or egg product (i.e., a food product that comprises a sulfur-comprising amino acid and that resembles a conventional egg or egg product and/or that can be used in place of a conventional egg or egg product). Non-limiting examples of eggs or egg products include whole egg (e.g., liquid whole egg, spray-dried whole egg, frozen whole egg), egg white (e.g., liquid egg white, spray-dried egg white, frozen egg white), egg yolk, egg dishes, egg soups, mixtures made with egg whites, mixtures made with egg substitutes, mayonnaise, custard, and salad dressings.

Resemblance of a substitute food product provided herein to a conventional food product may be due to any physical attribute, chemical/biological attribute, sensory attribute, and functional attribute, and any combination thereof.

The food product according to any of the above may be a pet food or animal feed.

The food product according to any of the above may be essentially free of any protein other than the protein comprising a sulfur-comprising amino acid comprised in the food product according to any of the above.

The food product according to any of the above may be essentially free of any recombinant protein other than the protein comprising a sulfur-comprising amino acid comprised in the food product according to any of the above.

The food product according to any of the above may be essentially free of a component found in a mammal-produced milk (e.g., cow milk, goat milk, sheep milk, human milk, buffalo milk, yak milk, camel milk, llama milk, alpaca milk, horse milk, donkey milk), or may comprise a lower concentration of at least one component found in a mammal-produced milk. Non-limiting examples of components found in mammal-derived milk include lactose, saturated fat, cholesterol, native milk proteins, and native milk lipids.

The food product according to any of the above may be essentially free of any milk protein other than the protein comprising a sulfur-comprising amino acid comprised in the food product according to any of the above.

The food product according to any of the above may be essentially free of a component obtained from an animal (i.e., a component that is native to an animal, including animal products [i.e., parts of an animal that are consumables or typically prepared for consumption by humans; e.g., animal meat, animal fat, animal blood], animal byproducts [i.e., products that are typically not consumable by themselves but are the byproducts of slaughtering animals for consumption; e.g., animal bones, animal carcasses, and constituents isolated therefrom], products produced by an animal [e.g., mammal-derived milk, chicken eggs, bee honey], and consumables produced therefrom [e.g., gelatin, rennet, whey proteins extracted from mammal-derived milk, casein extracted from mammal-derived milk, milk lipid extracted from mammal-derived milk, animal lipids, animal proteins]), or comprise 2% or less by mass of such component.

### Methods

In various aspects, provided herein is a method for producing a food product (e.g., substitute milk or barista beverage, high protein ready-to-drink beverage, frozen dessert mix, creamer, soft serve, shake mix) comprising between 1% and 12% by mass of an isolated β-lactoglobulin and/or recombinant β-lactoglobulin, wherein the method comprises:
a) obtaining the protein (e.g., optionally purified to at least 30%, at least 40%, at least 50%, at least 60%, at least 65%, at least 70%, at least 75%, at least 80%, at least 85%, at least 90%, at least 95%, at least 98%, or 100% purity) in a solution having a pH of between 6-8 (e.g., about pH 7; pH less than 7; pH less than 6.5; pH greater than 7; pH greater than 7.5; pH between 6 and 8, 7.8, 7.6, 7.4, 7.2, 7.0, 6.8, 6.6, 6.4, or 6.2; pH between 6.2 and 8, 7.8, 7.6, 7.4, 7.2, 7.0, 6.8, 6.6, or 6.4; pH between 6.4 and 8, 7.8, 7.6, 7.4, 7.2, 7.0, 6.8, or 6.6; pH between 6.6 and 8, 7.8, 7.6, 7.4, 7.2, 7.0, or 6.8; pH between 6.8 and 8, 7.8, 7.6, 7.4, 7.2, or 7.0; pH between 7.0 and 8, 7.8, 7.6, 7.4, or 7.2; pH between 7.2 and 8, 7.8, 7.6, or 7.4; pH between 7.4 and 8, 7.8, or 7.6; pH between 7.6 and 8, or 7.8; or pH between 7.8 and 8),
b) combining the protein with a suitable amount of an oxidizing agent (e.g., hydrogen peroxide at a molar ratio of less than 4 of hydrogen peroxide to β-lactoglobulin [e.g., molar ratio of less than 4, less than 3.5, less than 3, less than 2.5, less than 2, less than 1.5, or less than 1; between 0.5 and 3.8, 3.5, 3.2, 2.9, 2.6, 2.3, 2.0, 1.7, 1.4, 1.1, or 0.8; between 0.8 and 3.8, 3.5, 3.2, 2.9, 2.6, 2.3, 2.0, 1.7, 1.4, or 1.1; between 1.1 and 3.8, 3.5, 3.2, 2.9, 2.6, 2.3, 2.0, 1.7, or 1.4; between 1.4 and 3.8, 3.5, 3.2, 2.9, 2.6, 2.3, 2.0, or 1.7; between 1.7 and 3.8, 3.5, 3.2, 2.9, 2.6, 2.3, or 2.0; between 2.0 and 3.8, 3.5, 3.2, 2.9, 2.6, or 2.3; between 2.3 and 3.8, 3.5, 3.2, 2.9, or 2.6; between 2.6 and 3.8, 3.5, 3.2, or 2.9; between 2.9 and 3.8, 3.5, or 3.2; between 3.2 and 3.8, or 3.5; or between 3.5 and 3.8 of hydrogen peroxide to β-lactoglobulin]) and optional one or more other ingredients to obtain a mixture, and
c) subjecting the mixture to a process that results in production of the food product, respectively, wherein the process comprises heating the mixture at high temperature (e.g., temperatures used for pasteurization or UHT thermal processing, such as, for example, heating at between 70° C. and 150° C., 140° C., 130° C., 120° C., 110° C., 100° C., 90° C., or 80° C.; between 80° C. and 150° C., 140° C., 130° C., 120° C., 110° C., 100° C., or 90° C.; between 90° C. and 150° C., 140° C., 130° C., 120° C., 110° C., or 100° C.; between 100° C. and 150° C., 140° C., 130° C., 120° C., or 110° C.; between 110° C. and 150° C., 140° C., 130° C., or 120° C.; between 120° C. and 150° C., 140° C., or 130° C.; between 130° C. and 150° C., or 140° C.; or between 140° C. and 150° C. for at least 1 second; at least 3 seconds; at least 5 seconds; at least 10 seconds; at least 30 seconds; at least 1 minute; less than 1 minute; less than 45 seconds; less than 30 seconds; less than 15 seconds; less than 10 seconds; less than 5 seconds; between 1 second and 1 minute, 45 seconds, 30 seconds, 15 seconds, 10 seconds, or 5 seconds; between 5 seconds and 1 minute, 45 seconds, 30 seconds, 15 seconds, or 10 seconds; between 10 seconds and 1 minute, 45 seconds, 30 seconds, or 15 seconds; between 15 seconds and 1 minute, 45 seconds, or 30 seconds; between 30 seconds and 1 minute, or 45 seconds; or between 45 second and 1 minute).

The food product according to any of the above may be essentially free of another milk protein or another protein other than the isolated β-lactoglobulin and/or recombinant β-lactoglobulin.

### EXAMPLES

The following examples are included to illustrate specific embodiments of this disclosure. The techniques disclosed in the examples represent techniques discovered by the inventors to function well in the methods and processes of this disclosure; however, those of skill in the art should, in light of the present disclosure, appreciate that many changes can be made in the specific embodiments that are disclosed and still obtain a like or similar result without departing from the spirit and scope of the disclosure. Therefore, all matter set forth or shown in the examples is to be interpreted as illustrative and not in a limiting sense.

### Example 1: Production of Substitute Milk or Barista Beverage

A substitute milk or barista beverage was produced comprising between 2% and 7% by mass of recombinant β-lactoglobulin (produced in a recombinant Trichoderma reesei host cell; see, for example, PCT filing PCT/US2015/046428 filed Aug. 21, 2015, PCT filing PCT/US2017/48730 filed Aug. 25, 2017, and PCT/US2020/029392 filed Apr. 22, 2020), between 0.5% and 3.5% by mass of oil (e.g., coconut, sunflower, and/or canola oil), between 0% and 8% by mass of mtaltodextrin, between 0% and 2% by mass of sugar, between 0% and 2% by mass of inulin, between 0% and 1% by mass of gums, between 80% and 90% by mass of water, and between 0.1% and 1% by mass of buffering salt.

The dried ingredients were mixed with 50° C. water using a high shear blender for 5 minutes to hydrate the ingredients at an adjusted pH of 7+/−0.3. Melted oil was added while continuing to blend at low speed for 5 minutes. Hydrogen peroxide was added at between 0.8 and 2.8 molar ratio of hydrogen peroxide to β-lactoglobulin immediately prior to ultra-high temperature (UHT) thermal processing, which UHT thermal processing involved brief pre-heating of the mixture to 90° C. followed by direct steam injection to heat the mixture at between 135° C. and 147° C. for between 4 seconds and 6 seconds. The mixture was then flash cooled to between 60° C. and 80° C. to remove additional water, and homogenized with a dual-stage homogenizer running between 50-150 bar for the first stage and between 10-60 bar for the second stage. The resulting substitute milk or barista beverage was cooled to between 10° C. and 20° C., and then filled into desired containers. Sulfur off-odors were deemed to be faint to not detectable.

### Example 2: Production of Neutral pH, High Protein Ready-to-Drink (RTD) Beverage

A high protein RTD beverage was produced comprising between 2% and 11% by mass of recombinant β-lactoglobulin (produced in a recombinant Trichoderma reesei host cell; see, for example, PCT filing PCT/US2015/046428 filed Aug. 21, 2015, PCT filing PCT/US2017/48730 filed Aug. 25, 2017, and PCT/US2020/029392 filed Apr. 22, 2020), between 80% and 97% by mass of water, between 0.5% and 1% by mass of buffering salt, and less than 1.0% by mass of added flavors.

The dried ingredients were mixed with 50° C. water using a high shear blender for 5 minutes to hydrate the ingredients at an adjusted pH of 7+/−0.3. Hydrogen peroxide was added at between 0.8 and 2.4 molar ratio of hydrogen peroxide to β-lactoglobulin immediately prior to ultra-high temperature (UHT) thermal processing, which UHT thermal processing involved brief pre-heating of the mixture to 90° C. followed by direct steam injection to heat the mixture at between 135° C. and 147° C. for between 4 seconds and 6 seconds. The mixture was then flash cooled to between 60° C. and 80° C. to remove additional water, and then cooled to between 10° C. and 20° C., and filled into desired containers. Sulfur off-odors were deemed to be faint to not detectable.

### Example 3: Production of Substitute Aseptic Soft Serve/Shake Mix/Frozen Dessert Mix/Creamer

A substitute aseptic soft serve/shake mix/frozen dessert mix/creamer was produced comprising between 1% and 6% by mass of recombinant β-lactoglobulin (produced in a recombinant Trichoderma reesei host cell; see, for example, PCT filing PCT/US2015/046428 filed Aug. 21, 2015, PCT filing PCT/US2017/48730 filed Aug. 25, 2017, and PCT/US2020/029392 filed Apr. 22, 2020), between 0.5% and 18% by mass of oil (e.g., coconut, sunflower, and/or canola oil), between 0% and 8% by mass of maltodextrin, between 0% and 20% by mass of sugar or sugar substitutes, between 0% and 1% by mass of gums, between 60% and 80% by mass of water, and between 0.1% and 1% by mass of buffering salt.

The dried ingredients were mixed with 50° C. water using a high shear blender for 5 minutes to hydrate the ingredients at an adjusted pH of 7+/−0.3. Melted oil was added while continuing to blend at low speed for 5 minutes. Hydrogen peroxide was added at between 0.8 and 2.8 molar ratio of hydrogen peroxide to β-lactoglobulin immediately prior to ultra-high temperature (UHT) thermal processing, which UHT thermal processing involved brief preheating of the mixture to 90° C. followed by indirect-tubular heating to heat the mixture between 135° C. and 147° C. for between 4 seconds and 6 seconds. The mixture was then flash cooled to between 60° C. and 80° C., and homogenized with a dual-stage homogenizer running between 50-150 bar for the first stage and between 10-60 bar for the second stage. The resulting substitute mix was cooled to between 10° C. and 20° C., and then filled into desired containers. Sulfur off-odors were deemed to be faint to not detectable.

### Example 4: Production of Substitute HTST or Vat-Pasteurized Frozen Dairy Dessert Mix

A substitute pasteurized frozen dairy dessert mix was produced comprising between 1% and 6% by mass of recombinant β-lactoglobulin (produced in a recombinant Trichoderma reesei host cell; see, for example, PCT filing PCT/US2015/046428 filed Aug. 21, 2015, PCT filing PCT/US2017/48730 filed Aug. 25, 2017, and PCT/US2020/029392 filed Apr. 22, 2020), between 5% and 18% by mass of oil (e.g., coconut, sunflower, and/or canola oil), between 0% and 5% by mass of maltodextrin, between 0% and 20% by mass of sugar or sugar substitutes, between 0% and 1% by mass of gums, between 60% and 90% by mass of water, and between 0.1% and 1% by mass of buffering salt.

The dried ingredients were mixed with 20-50° C. water using a high shear blender for 5 minutes to hydrate the ingredients at an adjusted pH of 7+/−0.3. Melted oil was added while continuing to blend at low speed for 5 minutes. Hydrogen peroxide was added at between 0.8 and 2.8 molar ratio of hydrogen peroxide to β-lactoglobulin immediately prior to pasteurization (HTST or vat pasteurization). For HTST the mixture was heated to 82° C. for 25 seconds, for vat pasteurization the mixture was heated to 68-74° C. for between 25 seconds and 30 minutes. The pasteurized material was then homogenized with a dual-stage homogenizer running between 50-150 bar for the first stage and between 10-60 bar for the second stage. The resulting substitute mix was then cooled to below 10° C., and filled into desired containers. Sulfur off-odors were deemed to be faint to not detectable.

### Example 5: Effect of Molar Ratio of Hydrogen Peroxide to β-Lactoglobulin and High Temperature on Off-Flavor Production

Protein solutions were prepared comprising from 3.3 to 4.95 mM recombinant 3-lactoglobulin (produced in a recombinant Trichoderma reesei host cell; see, for example, PCT filing PCT/US2015/046428 filed Aug. 21, 2015, PCT filing PCT/US2017/48730 filed Aug. 25, 2017, and PCT/US2020/029392 filed Apr. 22, 2020) at an adjusted pH of 7.1+/−0.1. Hydrogen peroxide was added at the molar ratios shown in Table 1. Samples were subjected to thermal conditions in line with UHT processing by heating in a Perkin Elmer Rapid Viscoanalyzer with high temperature shear cell. The instrument was preheated to 141° C. Sample cells were filled with 60 g of sample, placed, and sealed in the sample chamber for about 2 min, before cooling to below 30° C. Once cooled, samples were evaluated qualitatively for the detection of sulfur off-odors such as cooked egg associated with hydrogen sulfide (H2S). At least two evaluators assessed samples. Each evaluator reported having at least 10 hours experience in the sensory evaluation of whey-protein based samples. Samples outcomes were based on detection of sulfur off aromas as either “no,” “faint,” or “strong.”

As shown in Table 1, at the conditions used, sulfur off-odors were deemed to be faint to not detectable.

TABLE 1
Sensory evaluation of protein solutions comprising
recombinant β-lactoglobulin (BLG) after UHT thermal
processing in presence of hydrogen peroxide

| | Protein | H2O2 | Molar ratio | Off- |
|---|---|---|---|---|
| Sample | (mM) | (mM) | of H2O2 to BLG | Flavor |
| WPI_086_1 | 3.30 | 2.40 | 0.73 | faint |
| WPI_086_2 | 3.30 | 3.00 | 0.91 | no |
| WPI_086_3 | 3.30 | 6.00 | 1.82 | no |
| WPI_086_4 | 4.95 | 3.60 | 0.73 | faint |
| WPI_086_5 | 4.95 | 4.00 | 0.81 | no |
| WPI_086_6 | 4.95 | 6.00 | 1.21 | no |

1. A method for producing a food product that comprises a substantial amount of a protein comprising a sulfur-comprising amino acid, wherein the method comprises:
a) obtaining the protein (e.g., in substantially purified form);
b) combining a substantial amount of the protein with a suitable amount of an oxidizing agent and optional one or more other ingredients to obtain a mixture; and
c) subjecting the mixture to a process that results in production of the food product, wherein the process comprises exposing the sulfur-comprising amino acid.

2. The method of claim 1, wherein the protein is a protein that was isolated from a natural source.

3. The method of claim 1, wherein the protein is a recombinant protein.

4. The method of claim 1, wherein the protein is a milk protein.

5. The method of claim 1, wherein the protein is an isolated or recombinant 3-lactoglobulin.

6. The method of claim 1, wherein the substantial amount is an amount that leads to production of an undesirable level of a volatile sulfur-containing compound.

7. The method of claim 1, wherein the protein is an isolated or recombinant β-lactoglobulin and the substantial amount is between 1% and 12% by mass of the isolated or recombinant β-lactoglobulin.

8. The method of claim 1, wherein the one or more other ingredients comprise a pH and/or ionic strength adjusting agent.

9. The method of claim 8, wherein the pH and/or ionic strength adjusting agent adjust pH of the mixture to between 6 and 8.

10. The method of claim 1, wherein the mixture further comprises more than 0.9% by mass of a lipid and an antioxidant.

11. The method of claim 1, wherein the oxidizing agents is selected from the group consisting of ascorbic acid, sodium ascorbate, azodicarbonamide, potassium bromate, potassium iodate, calcium iodate (lautarite), lipoxygenase, glucose oxidase, calcium peroxide, ammonium persulfate, potassium persulfate, ozone (e.g., sparged into the mixture), acetone peroxide, peracetic acid, chlorine, chlorine dioxide, benzoyl peroxide, rosemary extract, and hydrogen peroxide, dioxygen, and mixtures thereof.

12. The method of claim 11, wherein the oxidizing agents is a peroxide.

13. The method of claim 11, wherein the oxidizing agents is hydrogen peroxide.

14. The method of claim 1, wherein the protein is β-lactoglobulin, the oxidizing agents is hydrogen peroxide, and the suitable amount of the hydrogen peroxide is a molar ratio of less than 4.

15. The method of claim 14, wherein the suitable amount of the hydrogen peroxide is a molar ratio of between 0.5 and 3.8 of hydrogen peroxide to β-lactoglobulin.

16. The method of claim 14, wherein the suitable amount of the hydrogen peroxide is a molar ratio of between 0.5 and 3.0 of hydrogen peroxide to β-lactoglobulin.

17. The method of claim 1, wherein the exposing of the sulfur-comprising amino acid comprises high temperature heating.

18. The method of claim 17, wherein the high temperature heating is heating at between 70° C. and 150° C. for less than 1 minute.

19. The method of claim 18, wherein the high temperature heating is heating at between 70° C. and 100° C. for less than 1 minute.

20. The method of claim 18, wherein the high temperature heating is heating at between 130° C. and 150° C. for less than 10 seconds.

21. The method of any of claims 1 through 20, wherein the food product is a supplemented or substitute dairy product.

22. The method of claim 1, wherein the food product is selected from the group consisting of a milk or barista beverage, a high protein ready-to-drink beverage, a frozen dessert mix, and a soft serve/shake mix/frozen dessert mix/creamer.

23. The method of claim 1, wherein the food product is essentially free of another a protein other than the protein comprising a sulfur-comprising amino acid.

24. The method of claim 1, wherein the food product is essentially free of a milk protein other than the protein comprising a sulfur-comprising amino acid.
